(19) United States
(12) Patent Application Publication
(10) Pub. No.: US 2023/0000940 A1
(43) Pub. Date: Jan. 5, 2023

(54) Composition for prevention or treatment of Porcine epidemic diarrhea virus infection comprising curcuminoid and licorice extracts or fraction thereof

(71) Applicants: KOREA RESEARCH INSTITUTE OF BIOSCIENCE AND BIOTECHNOLOGY, Daejeon (KR); BIOTEN CO.,LTD, Jeongeup-si (KR)

(72) Inventors: Woo Song LEE, Daejeon (KR); Hyung Jun KWON, Daejeon (KR); Young Bae RYU, Daejeon (KR); Su-Jin PARK, Daejeon (KR); Ji Young PARK, Daejeon (KR); In Chul LEE, Daejeon (KR); Kyungsook JUNG, Daejeon (KR); Hyung Jae JEONG, Daejeon (KR)

(21) Appl. No.: 17/771,555

(22) PCT Filed: Oct. 23, 2020
(86) PCT No.: PCT/KR2020/014617

§ 371 Date: Jun. 9, 2022

(30) Foreign Application Priority Data
Oct. 25, 2019 (KR) 10-2019-0133852

(51) Int. Cl. A61K 36/484; A61K 31/121; A61K 36/9066; A61P 1/12

(52) U.S. Cl. CPC A61K 36/484; A61K 31/121; A61K 36/9066; A61P 1/12

(57) ABSTRACT

Provided are a pharmaceutical composition, a quasi-drug composition, a feed additive, a drinking water additive, a feed, and a drinking water for preventing, ameliorating, or treating porcine epidemic diarrhea (PED) virus infection, each including, as an active ingredient, a complex including a curcuminoid-based compound and a licorice extract or a fraction thereof.

### BACKGROUND OF THE INVENTION

### 1. Field of the Invention

The present invention relates to a pharmaceutical composition, a quasi-drug composition, a feed additive, a drinking water additive, a feed, and drinking water for preventing, ameliorating, or treating porcine epidemic diarrhea (PED) virus infection, each including, as an active ingredient, a complex including a curcuminoid-based compound and a licorice extract or a traction thereof.

### 2. Description of the Related Art

In order for functional substances to exhibit efficacy, it is essential that they be absorbed into the blood and maintain durability for a predetermined period of time. Amino acids, glucose, and vitamins, which are water-soluble substances, reach the heart through the liver, and fat granules produced by decomposition of fat, most phytochemicals, and fat-soluble vitamins, which are fat-soluble substances, are absorbed into the heart. Eventually, water-soluble and fat-soluble substances meet in the heart and are transported to cells throughout the body.

Regarding the research trends related thereto, research to develop water-soluble complexes using phospholipids for fat-soluble substances has been actively conducted. However, there is a limitation in that various problems such as decomposition of the materials occur. Accordingly, there has been a demand for a technique for maximizing the efficacy while maximizing the absorption rate by making the fat-soluble substances water-soluble.

Meanwhile, one of the main causes that lower the productivity of industrial animals is viral enteritis, which inevitably occurs more than once during the breeding period of piglets, and causes continuous economic loss by lowering the growth rate and causing death.

Recently, explosive outbreaks of variant porcine epidemic diarrhea (PED) domestically and abroad have caused enormous economic damage. Development of vaccines and therapeutic agents may be considered as a means to effectively control variant PED coronavirus. There have been no therapeutic agents for variant PED coronavirus in the world, and therefore, development of therapeutic agents is very important in terms of productivity and added value in the swine industry. Accordingly, it is necessary to develop therapeutic agents capable of effectively controlling the epidemic diarrhea virus in industrial animals.

The present inventors have conducted research on novel therapeutic agents for PED coronavirus infection, and they found that use of a curcuminoid-based compound together with a licorice extract exhibits a synergistic effect of increasing the absorption rate of the curcuminoid-based compound in the body to effectively prevent and treat PED coronavirus infection, thereby completing the present invention.

### SUMMARY OF THE INVENTION

An object of the present invention is to provide a pharmaceutical composition for preventing or treating porcine epidemic diarrhea (PED) virus infection, the pharmaceutical composition including, as an active ingredient, a complex including a curcuminoid-based compound or a pharmaceutically acceptable salt thereof; and a licorice extract or a fraction thereof.

Another object of the present invention is to provide a food composition for preventing or ameliorating PED virus infection, the food composition including, as an active ingredient, a complex including a curcuminoid-based compound or a pharmaceutically acceptable salt thereof; and a licorice extract or a fraction thereof.

Still another object of the present invention is to provide a quasi-drug composition for preventing or ameliorating PED virus infection, the quasi-drug composition including, as an active ingredient, a complex including a curcuminoid-based compound or a pharmaceutically acceptable salt thereof; and a licorice extract or a fraction thereof.

Still another object of the present invention is to provide a feed additive for preventing or ameliorating PED virus infection, the feed additive including, as an active ingredient, a complex including a curcuminoid-based compound or a pharmaceutically acceptable salt thereof; and a licorice extract or a fraction thereof.

Still another object of the present invention is to provide a feed including the feed additive.

Still another object of the present invention is to provide a drinking water additive for preventing or ameliorating PED virus infection, the drinking water additive including, as an active ingredient, a complex including a curcuminoid-based compound or a pharmaceutically acceptable salt thereof; and a licorice extract or a fraction thereof.

Still another object of the present invention is to provide drinking water including the drinking water additive.

### BRIEF DESCRIPTION OF THE DRAWINGS

FIG. 1 shows the effect of reducing porcine epidemic diarrhea (PED) coronavirus RNA by a curcumin-licorice saponin water-soluble complex;

FIG. 2 shows the target protein expression inhibitory activity of the complex on nucleoprotein (N protein) of PED coronavirus;

FIG. 3 shows the inhibitory activity of the complex on TNF-α;

FIG. 4 shows the inhibitory activity of the complex on IL-6;

FIG. 5 shows the inhibitory activity of the complex on IL-8;

FIG. 6 shows the survival rate of neonatal porcine epidemic virus animal models according to administration of the complex;

FIG. 7 shows the fecal consistency of neonatal porcine epidemic virus animal models according to administration of the complex:

FIG. 8 shows changes in the body weight of neonatal porcine epidemic virus animal models according to administration of the complex;

FIG. 9 shows the effect of reducing viral RNA in the feces of a complex-administered group;

FIG. 10 shows the effect of reducing viral RNA in the duodenum of the complex-administered group;

FIG. 11 shows the effect of reducing viral RNA in the jejunum of the complex-administered group;

FIG. 12 shows the effect of reducing viral RNA in the ileum of the complex-administered group:

FIG. 13 shows the effects of reducing TNF-α, IL-6, and IL-8 in the duodenum of the complex-administered group;

FIG. 14 shows the effects of reducing TNF-α, IL-6, and IL-8 in the jejunum of the complex-administered group;

FIG. 15 shows the effects of reducing TNF-α, IL-6, and IL-8 in the ileum of the complex-administered group;

FIG. 16 shows the effects of reducing TNF-α, IL-6, and IL-8 in the mesentery of the complex-administered group;

FIG. 17 shows results of histopathologic examination for the small intestine of the complex-administered group; and

FIG. 18 shows images showing a villi:crypt ratio on the specimens (upper) and a graph showing the villi:crypt ratio (lower).

### DETAILED DESCRIPTION OF THE PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

To achieve the above objects, one aspect of the present invention provides a pharmaceutical composition for preventing or treating porcine epidemic diarrhea (PED) virus infection, the pharmaceutical composition including, as an active ingredient, a complex including a curcuminoid-based compound represented by the following Formula 1 or a pharmaceutically acceptable salt thereof; and a licorice extract or a fraction thereof.

In the present invention, the active ingredient may include the complex including the curcuminoid-based compound represented by Formula 1 or the pharmaceutically acceptable salt thereof; and the licorice extract or the fraction thereof, as well as a mixture of the curcuminoid-based compound represented by Formula 1 or the pharmaceutically acceptable salt thereof; and the licorice extract or the fraction thereof.

As used herein, the term “porcine epidemic diarrhea (PED)” refers to a contagious swine disease caused by PED virus (PEDV). The characteristic gross clinical symptoms include depression, anorexia immediately before/after diarrhea, vomiting, and dehydration and weight loss due to severe watery diarrhea. Characteristic autopsy findings are that the small intestine is filled with gas and fluid, the serous membrane becomes thinner, and the intestinal mucosal folds disappears. PEDV is a type of coronavirus, and in the present invention, the PEDV may be used interchangeably with PED virus or PED coronavirus.

In the present invention, the curcuminoid-based compound may be represented by the following Formula 1, but is not limited thereto.

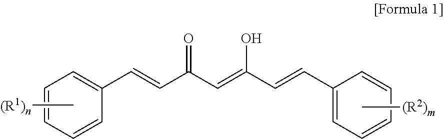

R¹ and R² are each independently hydrogen, a hydroxy group, or a C₁-C₁₀ alkoxy group, and n and m are 1≤n≤55 and 1≤m≤5.

Specifically, the curcuminoid-based compound represented by Formula 1 may be a compound represented by any one selected from Formulae 2 to 4, or a mixture thereof, but is not limited thereto.

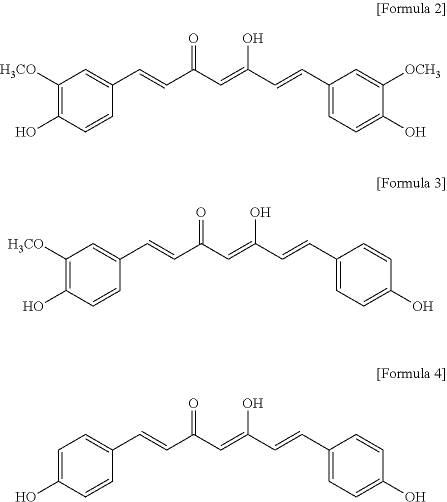 [SMILES: COc1cc(/C=C/C(=O)/C=C(O)/C=C/c2ccc(O)c(OC)c2)ccc1O ; COc1cc(/C=C/C(=O)/C=C(O)/C=C/c2ccc(O)cc2)ccc1O ; O=C(/C=C(O)/C=C/c1ccc(O)cc1)/C=C/c1ccc(O)cc1]

The compound of Formula 2 may be curcumin, and the compounds of Formula 3 and Formula 4 may be derivatives of curcumin. The compound of Formula 3 may be called demethoxycurcumin, and the compound of Formula 4 may be called bisdemethoxycurcumin.

With respect to the objects of the present invention, any compound belonging to the curcuminoid-based compound, as well as the curcumin, demethoxycurcumin, and bisdemethoxycurcumin, may be used without limitation. It is apparent to those skilled in the art that derivatives or isomers of the curcumin, demethoxycurcumin, and bisdemethoxycurcumin may also be used.

As used herein, the term “derivative” refers to a compound obtained by substituting a part of the structure of the compound with another atom or atom group.

As used herein, the term “isomer” refers to a compound with the same molecular formula, but different connectivity or spatial arrangement of constituent atoms in the molecule. The isomer includes, for example, structural isomers and stereoisomers.

In the present invention, the curcuminoid-based compound represented by Formula 1 may be isolated from turmeric, but is not limited thereto.

As used herein, the term “pharmaceutically acceptable salt” means any salt of the compound of the present invention that is physiologically acceptable to a target individual, and a formulation of the compound, which does not abrogate the biological activity and properties of the compound without causing significant irritation to an organism to which the compound is administered, is preferred. The pharmaceutical salts may include acid addition salts which may form non-toxic acid addition salts containing pharmaceutically acceptable anions, for example, inorganic acids such as hydrochloric acid, sulfuric acid, nitric acid, phosphoric acid, hydrobromic acid, hydriodic acid, etc.; organic carbonic acids such as tartaric acid, formic acid, citric acid, acetic acid, trichloroacetic acid, trifluoroacetic acid, gluconic acid, benzoic acid, lactic acid, fumaric acid, maleic acid, salicylic acid, etc.; sulfonic acids such as methanesulfonic acid, ethanesulfonic acid, benzenesulfonic acid, p-toluenesulfonic acid, etc. For example, the pharmaceutically acceptable carboxylic acid salt includes metal salts or alkaline earth metal salts formed with lithium, sodium, potassium, calcium, magnesium, etc., amino acid salts such as lysine, arginine, guanidine, etc., organic salts such as dicyclohexylamine, N-methyl-D-glucamine, tris(hydroxymethyl)methylamine, diethanolamine, choline, triethylamine, etc.

As used herein, the term “licorice” refers to the root of the plant of the genus Glycyrrhiza, which is known to stop coughing, relieve fever, soothe the stomach, and relieve emergency situations.

As used herein, the term “extract” includes a liquid extract itself and an extract of any formulation, which may be formed using the liquid extract, such as a liquid extract obtained by extracting the licorice, a diluted or concentrated solution of the liquid extract, a dry product obtained by drying the liquid extract, a crude purified product or purified product of the liquid extract, or a mixture thereof.

As a method of preparing the licorice extract, a common extraction method in the art, such as ultrasonic extraction, filtration, reflux extraction, etc., may be used. The licorice extract may be preferably an extract obtained by extracting a dry product of licorice obtained by grinding licorice, from which foreign substances are removed by washing and drying, with a water, a C₁-C₄ alcohol, or a mixed solvent thereof, more preferably an extract obtained by extracting with a C₁-C₄ alcohol, and most preferably an extract obtained by extracting with methanol or ethanol. In this regard, the extraction solvent is preferably used in an amount of 2 time to 20 times the dry weight of licorice. For example, the dry product of licorice is finely cut, and then placed in an extraction vessel. A C₁-C₄ lower alcohol or a mixed solvent thereof, preferably methanol or ethanol is added thereto, and left at room temperature for a predetermined period of time, and then filtered to obtain the alcohol extract. In this regard, the extraction is preferably left at room temperature for 1 week, and subsequently, a method such as concentration or freeze-drying may be additionally performed. Alternatively, a commercially available licorice extract may be purchased and used.

The main component of the licorice extract is glycyrrhizinic acid, which is a white or colorless crystalline powder. The glycyrrhizinic acid may also be referred to as licorice saponin. In the present invention, the licorice extract may include 7% or more of licorice saponin, which is glycyrrhizinic acid prepared in the form of licorice extract or powder, but is not limited thereto.

As used herein, the term “fraction” refers to a product obtained by a fractionation method of separating a specific component or a specific group from a mixture containing various components. With regard to the licorice fraction, a polar solvent fraction and a non-polar solvent fraction may be obtained respectively by, for example, suspending the licorice extract in water and then performing fractionation using a non-polar solvent such as hexane or ethyl acetate. Specifically, the fraction may be obtained by suspending the licorice crude extract in distilled water, and then adding a non-polar solvent such as hexane or ethyl acetate with a volume of about once to 100 times, preferably about once to 5 times the volume of the suspension, and extracting and separating a non-polar solvent soluble layer over once to 10 times, preferably twice to five times, but is not limited thereto. Further, a common fractionation process may be additionally performed (Harbome, J. B. Phytochemical methods: A guide to modern techniques of plant analysis, 3rd Ed. pp. 6-7, 1998).

In order for functional substances to exhibit efficacy in the body, it is essential that they be absorbed into the blood and maintain durability for a predetermined period of time. Accordingly, for water-solubilization of curcumin, which is a functionally poorly soluble substance, in one exemplary embodiment of the present invention, curcumin was mixed with the licorice extract, and a microwave stirring extractor (Korean Patent No. 10-1894087) was used to prepare a curcumin-licorice saponin water-soluble complex.

As used herein, the term “water-solubilization” refers to a phenomenon in which solubility of a poorly water-soluble substance is increased by the presence of a substance such as surfactants. As a method for the water-solubilization, a method of solubilizing oil-soluble vitamins or hormones to make them water-soluble or a method of accelerating an emulsion polymerization has been widely applied. In the present invention, the licorice extract is used as a water solubilizing agent, and the licorice extract of the present invention is mixed with the poorly water-soluble curcuminoid-based compound to form a curcuminoid-based compound-licorice saponin water-soluble complex having a structure, in which the curcuminoid-based compound is easily soluble in water (or blood).

A weight ratio of the curcuminoid-based compound or the pharmaceutically acceptable salt thereof: the licorice extract or the fraction thereof included in the complex may be 1:1 to 1:400, 1:10 to 1:400, 1:20 to 1:400, or 1:30 to 1:100, specifically 1:50 to 1:70, but is not limited thereto.

As used herein, the term “preventing” means all of the actions by which the occurrence of PED virus infection is restrained or retarded by the administration of the composition, and the term “treating” means all of the actions by which symptoms of PED virus infection have taken a turn for the better or been modified favorably by the administration of the composition.

The pharmaceutical composition of the present invention may further include an appropriate carrier, excipient, or diluent commonly used in the preparation of the pharmaceutical composition. At this time, the amount of the active ingredient included in the pharmaceutical composition may be, but is not particularly limited to, 0.0001% by weight to 10% by weight, preferably 0.001% by weight to 1% by weight, based on the total weight of the composition.

The pharmaceutical composition may have any one formulation selected from the group consisting of tablets, pills, powders, granules, capsules, suspensions, solutions for internal use, emulsions, syrups, sterilized aqueous solutions, non-aqueous solvents, suspensions, emulsions, lyophilized preparations, and suppositories, and may have various formulations for oral or parenteral administration. When formulated, the composition of the present invention may be prepared using commonly used diluents or excipients, such as fillers, extenders, binders, wetting agents, disintegrants, surfactants, etc. Solid formulations for oral administration include tablets, pills, powders, granules, capsules, etc., and such solid formulations are prepared by mixing one or more compounds with one or more excipients, for example, starch, calcium carbonate, sucrose, lactose, gelatin, etc. In addition to simple excipients, lubricants such as magnesium stearate, talc, etc. may also be used. Liquid formulations for oral administration include suspensions, solutions for internal use, emulsions, syrup, etc., and may include various excipients, for example, wetting agents, flavoring agents, aromatics, preservatives, etc., in addition to water and liquid paraffin, which are simple diluents frequently used. Formulations for parenteral administration include sterilized aqueous solutions, non-aqueous solutions, suspensions, emulsions, lyophilized preparations, and suppositories. As non-aqueous solvents or suspending agents, propylene glycol, polyethylene glycol, plant oils such as olive oil, injectable esters such as ethyl oleate, etc. may be used. As a base of suppositories, witepsol, Macrogol. Tween 61, cacao butter, laurin fat, glycerogelatin, etc. may be used.

The pharmaceutical composition of the present invention may be for oral administration, but is not limited thereto.

The composition of the present invention may be administered in a pharmaceutically effective amount.

As used herein, the term “pharmaceutically effective amount” refers to an amount sufficient to treat diseases at a reasonable benefit/risk ratio applicable to any medical treatment. The effective dosage level may be determined depending on factors including a subject's type and severity, age and sex, the type of disease, the activity of a drug, sensitivity to the drug, the administration time and route, excretion rate, the duration of treatment, drugs used concurrently, and other factors known in the medical field. The pharmaceutical composition of the present invention may be administered individually or in combination with other therapeutic agents, and may be administered sequentially or simultaneously with traditional therapeutic agents. The composition may be administered in a single- or multiple-dosage form. It is important to administer the composition in the minimum amount that exhibits the maximum effect without causing side effects, in view of all the above-described factors, and this amount may be easily determined by a person skilled in the art. A preferred administration dosage of the composition of the present invention may vary depending on a patient's condition, body weight, severity of disease, the type of a drug, administration route, and period. However, for preferred effects, the composition of the present invention may be administered at a daily dose of 0.0001 mg/kg to 500 mg/kg, preferably 0.001 mg/kg to 200 mg/kg. The composition may be administered once or several times a day. The composition may be administered to various mammals such as rats, livestock, humans, etc. via various routes. All modes of administration common in the art are included without limitation, for example, orally, rectally, or by intravenous, intramuscular, subcutaneous, intrauterine, or intracerebroventricular injection. Specifically, the composition may be orally administrated, but is not limited thereto.

Further, the pharmaceutical composition of the present invention may be used in the form of veterinary medicines as well as medicines applied to humans. Here, the animal is a concept including livestock and pets.

In the present invention, the complex including the curcuminoid-based compound represented by Formula 1 or the pharmaceutically acceptable salt thereof; and the licorice extract or the fraction thereof may be prepared by a method including the steps of a) mixing a first composition including the curcuminoid-based compound represented by Formula 1 or the pharmaceutically acceptable salt thereof as an active ingredient; and a second composition including the licorice extract or the fraction thereof as an active ingredient; and b) treating the mixture with microwave.

In the step a), a weight ratio of the first composition and the second composition during mixing may be 1:1 to 1:400, 1:10 to 1:400, 1′20 to 1:400, 1:30 to 1:100, specifically, 1:50 to 1:70, but is not limited thereto.

In the step b), the method of treating with microwave may be, but is not limited to, stirring extraction at 2,200 W to 24,000 W, preferably 2,200 W to 12,000 W for 10 min to 120 min, preferably 10 min to 60 min using a microwave stirring extractor (Korean Patent No. 10-1894087).

As used herein, the term “microwave” refers to an alternating signal with a frequency band between 300 MHz and 300 GHz or a wavelength between 1 m and 1 mm. In addition, the method may further include the step of purifying with a filter paper after completing the extraction, but is not limited thereto.

Another aspect of the present invention provides a food composition for preventing or ameliorating PED virus infection, the food composition including, as an active ingredient, the complex including the curcuminoid-based compound represented by Formula 1 or the pharmaceutically acceptable salt thereof; and the licorice extract or the fraction thereof.

The curcuminoid-based compound, the licorice extract or fraction thereof, the PED virus are the same as described above.

Specifically, the curcuminoid-based compound represented by Formula 1 may be any one of curcumin, demethoxycurcumin, and bisdemethoxycurcumin, or a mixture thereof, but is not limited thereto.

The active ingredient of the present invention may be added to the food composition for the purpose of preventing or ameliorating PED virus infection. When the active ingredient of the present invention is used as a food additive, the active ingredient may be added as it is, or may be used together with another food or ingredient, and may be appropriately used according to a common method. The mixing amount of the active ingredient may be appropriately determined according to the purpose of use, and the amount of the active ingredient included in the food composition may be, but is not particularly to, 0.0001% by weight to 10% by weight, preferably 0.001% by weight to 1% by weight, based on the total weight of the composition.

There is no particular limitation on the kind of food of the present invention. Examples of the foods, to which the active ingredient may be added, may include meats, sausages, bread, chocolate, candies, snack, confectionery, pizza, instant noodles, other noodles, gum, dairy products including ice cream, various soups, beverages, teas, drinks, alcoholic beverages and multivitamin preparations. The foods may include all foods in the ordinary acceptation of the term, and may include foods used as feeds for animals.

In addition, the food composition of the present invention may include various nutrients, vitamins, electrolytes, flavoring agents, colorants, pectic acid and salts thereof, alginic acid and salts thereof, organic acids, protective colloidal thickeners, pH adjusters, stabilizers, preservatives, glycerin, alcohol, carbonating agents used in carbonated drinks, etc. Additionally, the food composition of the present invention may include fruit flesh for the preparation of natural fruit juices, fruit juice beverages and vegetable juices. Further, the food may be prepared in the form of tablets, granules, powders, capsules, liquid solutions, pills according to known preparation methods. There is no particular limitation on other ingredients, except that the active ingredient according to the present invention is included, and a variety of common flavoring agents or natural carbohydrates may be included as additional ingredients.

Still another object of the present invention provides a quasi-drug composition for preventing or ameliorating PED virus infection, the quasi-drug composition including, as an active ingredient, the complex including the curcuminoid-based compound represented by Formula 1 or the pharmaceutically acceptable salt thereof; and the licorice extract or the fraction thereof.

The curcuminoid-based compound, the licorice extract or fraction thereof, the PED virus are the same as described above.

Specifically, the curcuminoid-based compound represented by Formula 1 may be any one of curcumin, demethoxycurcumin, and bisdemethoxycurcumin, or a mixture thereof, but is not limited thereto.

The active ingredient of the present invention may be added to the quasi-drug composition for the purpose of preventing or ameliorating PED virus infection.

As used herein, the term “quasi-drug” refers to defined as articles made from fiber, rubber, or similar materials used for the purpose of curing, alleviating, treating, or preventing human or animal diseases; articles, other than instruments, machines or the like, which have a mild action on or have no direct influence on the human body; and articles, falling within the range of agents used to sterilize, kill insects and for similar purposes for the prevention of infection, and the quasi-drug does not include products used for the purposes of diagnosis, medical care, alleviation, treatment, or prevention of human or animal diseases, excluding appliances, machinery and equipment; or products, other than appliances, machinery, or equipment, used for the purpose of exerting pharmacological effects upon the structure or functions of humans or animals. The quasi-drugs may include external preparation for the skin and personal hygiene products.

The external preparation for the skin may be preferably prepared in the form of, but is not particularly limited to, an ointment, lotion, spray, patch, cream, powder, suspension, gel formulation, or gel. The above personal hygiene products may be, but are not particularly limited to, preferably soaps, cosmetics, wet wipes, toilet paper, shampoos, skin creams, face creams, toothpastes, lipsticks, perfumes, makeup foundations, cheek touch, mascaras, eye shadows, sunscreen lotions, hair care products, air freshener gels, or cleansing gels. In addition, another example of the quasi-drug composition of the present invention includes disinfectant cleaners, shower forms, mouthwashes, wet wipes, detergent soaps, hand washes, humidifier fillers, masks, ointments, or filter fillers.

When the active ingredient of the present invention is added to the quasi-drug composition for the purpose of preventing or ameliorating PED virus infection, the active ingredient may be added as it is, or may be used together with another quasi-drug component, and may be appropriately used according to a common method. The mixing amount of the active ingredient may be appropriately determined according to the purpose of use, and the amount of the active ingredient included in the quasi-drug composition may be, but is not particularly to, 0.0001% by weight to 10% by weight, preferably 0.001% by weight to 1% by weight, based on the total weight of the composition.

Still another object of the present invention provides a feed additive or drinking water additive for preventing or ameliorating PED virus infection, the feed additive or drinking water additive including, as an active ingredient, the complex including the curcuminoid-based compound represented by Formula 1 or the pharmaceutically acceptable salt thereof; and the licorice extract or the fraction thereof.

The curcuminoid-based compound, the licorice extract or fraction thereof, the PED virus are the same as described above.

Specifically, the curcuminoid-based compound represented by Formula 1 may be any one of curcumin, demethoxycurcumin, and bisdemethoxycurcumin, or a mixture thereof, but is not limited thereto.

As used herein, the term “feed additive” collectively refers to substances added in trace amounts to feeds for nutritional or specific purposes. In the present invention, the feed additive refers to a substance added for the purpose of preventing or ameliorating PED virus infection.

As used herein, the term “feed” refers to a substance providing organic or inorganic nutrients which are necessary to maintain a subject's life or to raise the subject. The feed may include nutrients such as energy, proteins, lipids, vitamins, minerals, etc. which are required by a subject which consumes the feed, but is not particularly limited thereto. The feed may be a plant-based feed such as grains, nuts, food by-products, algae, fibers, oils, starch, meals, grain by-products, etc., or an animal-based feed such as proteins, inorganic substances, fats, minerals, single-cell proteins, zooplanktons, fish meals, etc.

The subject refers to a subject to be raised, and may include any living organism without limitation, as long as it is able to intake the feed of the present disclosure. For example, the subject is a concept including livestock and pets.

Further, in addition to the feed additive of the present disclosure, the feed may include additional feed additives to promote a subject's growth and to prevent diseases, such as amino acids, vitamins, enzymes, flavoring agents, non-protein nitrogenous compounds, silicates, buffer, extracting agents, oligosaccharides, etc.

The feed additive of the present invention may further include a binder, an emulsifier, a preservative, etc., which are added to prevent deterioration in quality, and may further include amino acids, vitamins, enzymes, probiotics, flavoring agents, non-protein nitrogenous compounds, silicates, buffer, colorants, extracting agents, oligosaccharides, etc., which are added to increase effects. In addition, the feed additive may include other teed mixture, etc., but is not limited thereto.

As used herein, the term “drinking water additive” collectively refers to substances added in trace amounts to drinking water for animals for nutritional or specific purposes. In the present invention, it refers to a substance added for the purpose of preventing or ameliorating PED virus infection. Here, the animal is a concept including livestock and pets.

Still another object of the present invention provides a feed or drinking water including the feed additive or drinking water additive for preventing or ameliorating PED virus infection, the feed additive or drinking water additive including, as an active ingredient, the complex including the curcuminoid-based compound represented by Formula 1 or the pharmaceutically acceptable salt thereof; and the licorice extract or the fraction thereof.

The curcuminoid-based compound, the licorice extract or fraction thereof, the PED virus, the feed additive, the feed, and the drinking water additive are the same as described above.

Hereinafter, the present disclosure will be described in more detail with reference to exemplary embodiments. However, these exemplary embodiments are only for illustrating the present disclosure, and the scope of the present disclosure is not intended to be limited by these exemplary embodiments.

### Example 1: Method of Preparing Curcumin-Licorice Saponin Water-Soluble Complex and Powder

As curcumin, which is a curcuminoid-based compound, Indian curcumin with a purity of about 95% or more was used. A licorice extract (DAEPYUNG CO., LTD., Korea) is an extract including 7% or more of licorice saponin which is glycyrrhizinic acid prepared in the form of licorice extract or powder.

25 kg of the licorice extract was dissolved in 125 L of water, and then 375 g of curcumin was added and mixed therewith. Thereafter, the mixture was placed in a microwave stirring extractor (Korean Patent No. 10-1894087), and extracted with stirring at 12,000 W for 60 minutes. When the extraction was completed, the extract was purified with a filter paper to prepare a liquid-type curcumin-licorice saponin water-soluble complex. Thereafter, a powder of the curcumin-licorice saponin water-soluble complex (hereinafter referred to as a complex) was obtained through freeze-drying under vacuum.

### Example 2: Evaluation of In Vitro Efficacy of Complex on PED Coronavirus

2-1. Effect of Reducing PED Coronaviral RNA

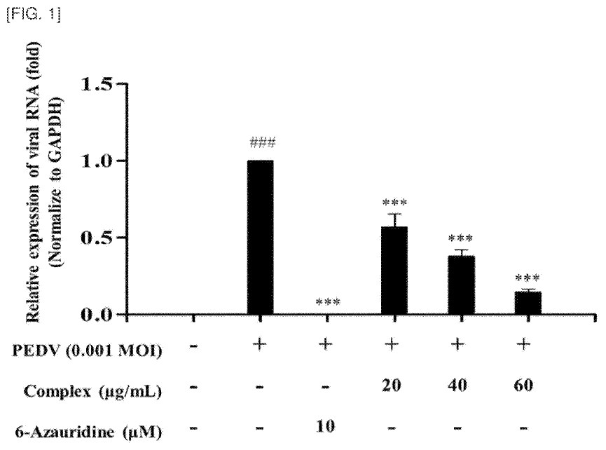

To investigate the effect of reducing RNA of porcine epidemic diarrhea (PED) coronavirus by the complex, Vero cells were infected with 0.001 MOI of PED coronavirus for 1 hour. 24 hours after treatment with the complex at each concentration (20 μg/mL, 40 μg/mL, 60 μg/mL), the results were examined by real-time PCR. With regard to the ability to reduce viral RNA, the complex-administered group of 20 μg/mL, 40 μg/mL, or 60 μg/mL concentration showed 1.7-fold, 2.6-fold, or 6.6-fold viral RNA reduction in a concentration-dependent manner, respectively (FIG. 1), as compared with the virus-infected group.

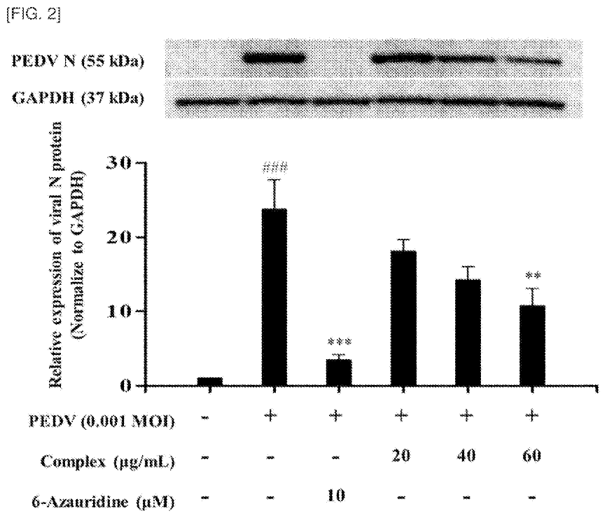

To investigate the target protein expression inhibitory effect of the complex on the nucleoprotein (N protein) of the PED coronavirus, Vero cells were infected with 0.001 MOI of PED coronavirus for 1 hour. 24 hours after treatment with the complex at each concentration, the results were examined. As compared with the virus-infected group, the positive control 6-azauridine showed 6.8-fold inhibitory effect at 10 μM, and the complex-administered group of 20 μg/mL, 40 μg/mL, or 60 μg/mL concentration showed 1.3-fold, 1.6-fold, or 2.2-fold nucleoprotein synthesis inhibition in a concentration-dependent manner, respectively (FIG. 2).

2-2. Effect of Reducing Anti-Inflammatory Factors in PED Coronavirus Infection

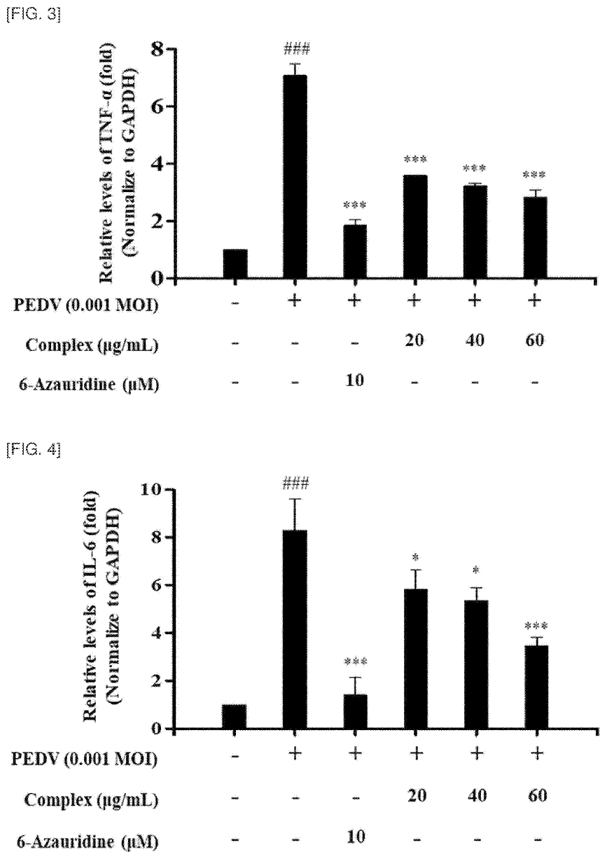

To investigate the inhibitory effect of the complex on TNF-α, which is a pro-inflammatory cytokine involved in promoting inflammation, virus infection was performed in the same manner as in Example 2-1, followed by treatment with the complex. As compared with the virus-infected group, the positive control 6-azauridine showed 3.8-fold inhibitory effect at 10 μM, and the complex-administered group of 20 μg/mL, 40 μg/mL, or 60 μg/mL concentration showed 1.9-fold, 2.2-fold, or 2.5-fold anti-inflammatory activity, respectively (FIG. 3).

To investigate the inhibitory effect of the complex on IL-6, which is a pro-inflammatory cytokine involved in promoting inflammation, virus infection was performed in the same manner as in Example 2-1, followed by treatment with the complex. As compared with the virus-infected group, the positive control 6-azauridine showed 5.8-fold inhibitory effect at 10 μM, and the complex-administered group of 20 μg/mL, 40 μg/mL, or 60 μg/mL concentration showed 1.4-fold, 1.5-fold, or 2.4-fold anti-inflammatory activity in a concentration-dependent manner, respectively (FIG. 4).

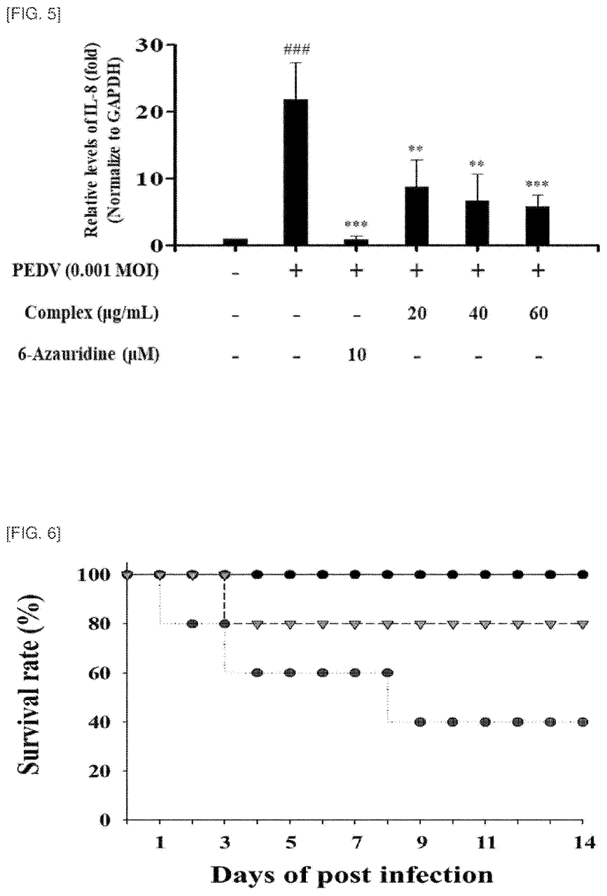

To investigate the inhibitory effect of the complex on IL-8, which is a pro-inflammatory cytokine involved in promoting inflammation, virus infection was performed in the same manner as in Example 2-1, followed by treatment with the complex. As compared with the virus-infected group, the positive control 6-azauridine showed 25.4-fold inhibitory effect at 10 μM, and the complex-administered group of 20 μg/mL, 40 μg/mL, or 60 μg/mL concentration showed 3.7-fold, 4.1-fold, or 6.4-fold high anti-inflammatory activity in a concentration-dependent manner, respectively (FIG. 5).

### Example 3: Construction of Neonatal Porcine Epidemic Virus Animal Model and Evaluation of Efficacy of Complex

3-1. Construction of Neonatal Porcine Epidemic Virus Animal Model

When grouping was performing using 3-day-old neonatal piglets of mothers who were not vaccinated against PED virus, piglets with the similar average body weight were classified and fed milk replacer. A negative control group, which was not inoculated with the virus and not administered with the complex, was orally administered with an equal amount of alpha MEM medium (Alpha Modification of Eagles MEM). A complex-administered experimental group (5n) was orally administered with 200 mg/kg/day of the complex for 5 days, and then lactating piglets were orally challenged with 2 mL of 1×10² PFU/mL PEDV (Aram strain, Choong Ang Vaccine Laboratories Co., Ltd.), and raised together with diarrhea-induced (PID2) piglet (1n) to construct diarrhea-causing epidemic models, respectively.

3-2. Survival Results

The evaluation groups consisted of a mock-infected group, a virus-infected group, and an experimental group (raised together, and 200 mg/kg/day administered). Piglets (1n) of the experimental group were raised together with mock-infected piglets (5n), and survival rates of epidemic diarrhea models according to administration for 14 days were measured.

As a result, the mock-infected group survived 100% until the end of the test period, and the virus-infected group showed a survival rate of 40%. In contrast, the complex-administered group showed a survival rate of 80%, indicating 2-fold increase in the survival rate, as compared with the virus-infected group (FIG. 6).

3-3. Observation of Fecal Consistency and Results

During the course of the experiment, fecal consistency was observed every day, and the fecal score of each individual was recorded and averaged. Fecal consistency was scored as follows: normal (0), pasty (1), semi-liquid (2), and liquid (3).

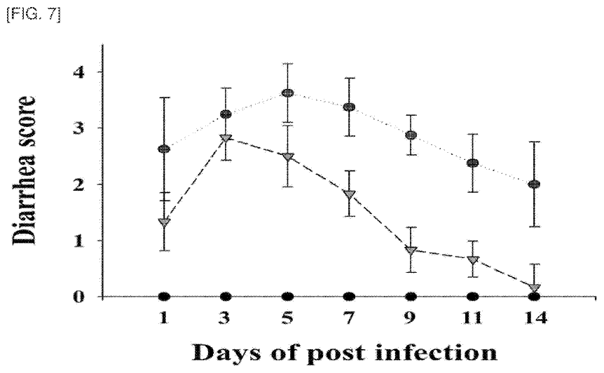

As a result, a positive control group (virus), which was inoculated with the virus and not administered with the complex, showed a fecal score of 3 until 10 days after inoculation. In contrast, the experimental group administered with 200 mg/kg/day of the complex showed the same fecal consistency as the normal control group, day 10 after virus inoculation, indicating that the complex had very high efficacy of ameliorating and treating porcine epidemic diarrhea (FIG. 7).

3-4. Body Weight

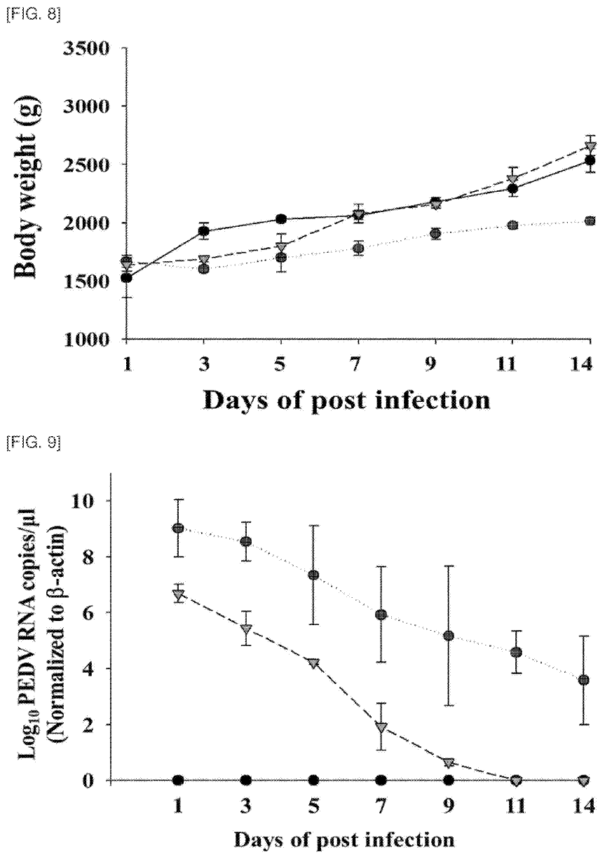

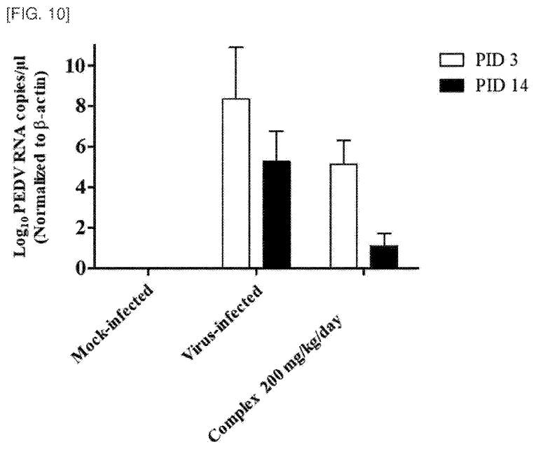

Changes in the body weight according to the complex administration were examined for 14 days in the epidemic model of Example 3-2. As a result, the mock-infected group showed a 1.5-fold increase in the body weight from the start to the end of the test period, and the virus-infected group showed no significant difference in the body weight. In contrast, the complex-administered group showed a 1.5-fold increase in the body weight, indicating that the body weight was increased even though infected (FIG. 8).

3-5. Effect of Reducing Viral RNA in Feces/Small Intestine by Complex

3-5-1. Feces

Viral RNA in the feces tended to gradually decrease from day 3 after infection (PID3) to day 14 after infection (PID14) in the virus-infected group and the experimental group (raised together and 200 mg/kg/day administered), except for the mock-infected group. In the virus-infected group, virus was detected until the end of the test period. In contrast, in the complex-administered group, viral RNA was not detected from day 11 after infection (PID11) to the end date, indicating the high efficacy of ameliorating and treating porcine epidemic diarrhea (FIG. 9).

3-5-2. Duodenum

Viral RNA in the duodenum tended to gradually decrease from day 3 after infection (PID3) to day 14 after infection (PID14) in the virus-infected group and the experimental group (raised together and 200 mg/kg/day administered), except for the mock-infected group. At the end of the test period (PID14), viral RNA in the duodenum showed an about 5-fold decrease in the complex-administered group, as compared with the virus-infected group, indicating the high efficacy of ameliorating and treating porcine epidemic diarrhea (FIG. 10).

3-5-3. Jejunum

Viral RNA in the jejunum tended to gradually decrease from day 3 after infection (PID3) to day 14 after infection (PID14) in the virus-infected group and the experimental group (raised together and 200 mg/kg/day administered), except for the mock-infected group. At the end of the test period (PID14), viral RNA in the jejunum showed an about 6-fold decrease in the complex-administered group, as compared with the virus-infected group, indicating the high efficacy of ameliorating and treating porcine epidemic diarrhea (FIG. 11).

3-5-4. Ileum

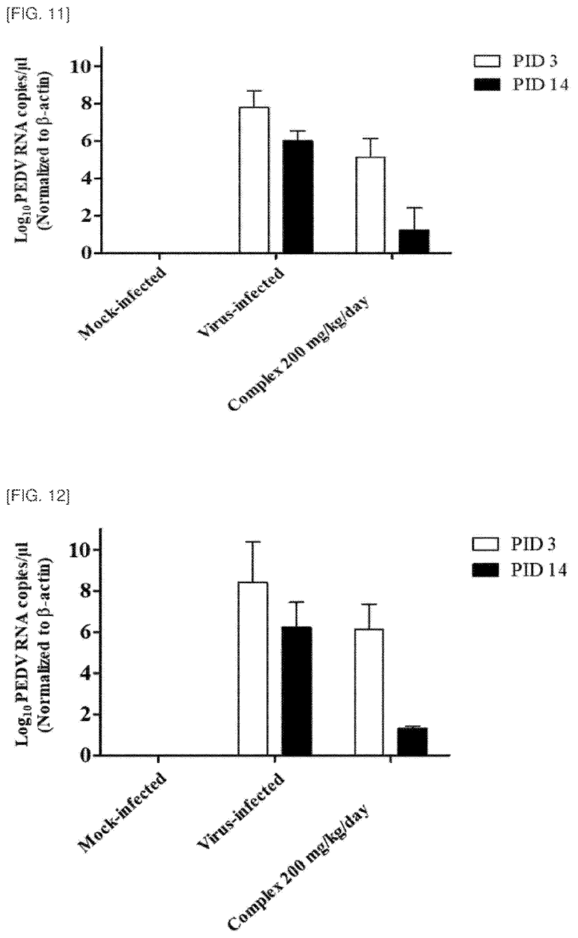

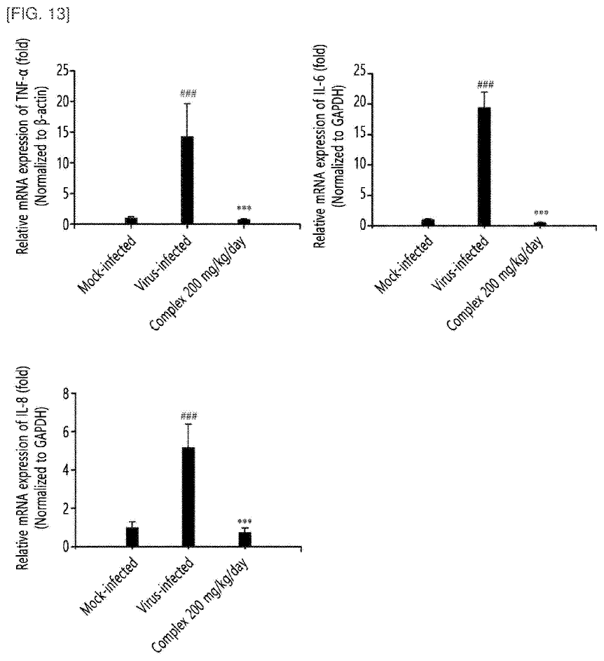

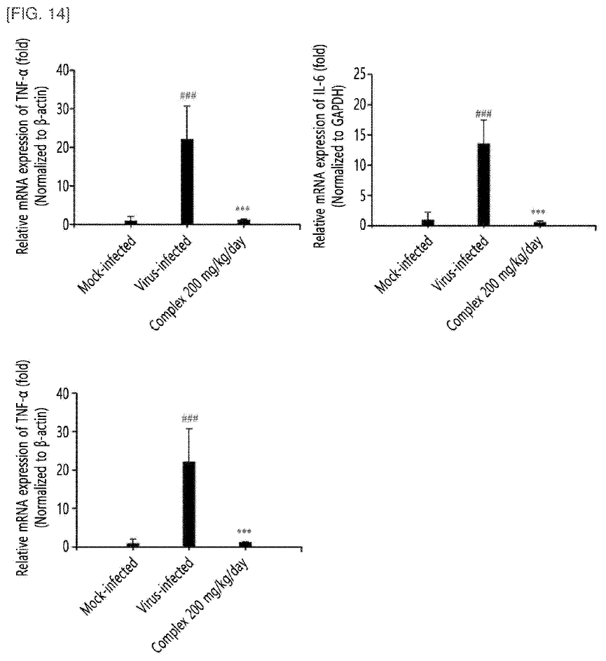

Viral RNA in the ileum tended to gradually decrease from day 3 after infection (PID3) to day 14 after infection (PID14) in the virus-infected group and the experimental group (raised together and 200 mg/kg/day administered), except for the mock-infected group. At the end of the test period (PID14), viral RNA in the ileum showed an about 6-fold decrease in the complex-administered group, as compared with the virus-infected group, indicating the high efficacy of ameliorating and treating porcine epidemic diarrhea (FIG. 12).

3-6. Effects of Reducing Pro-Inflammatory Factors in Small Intestine/Mesentery by Complex

3-6-1. Duodenum

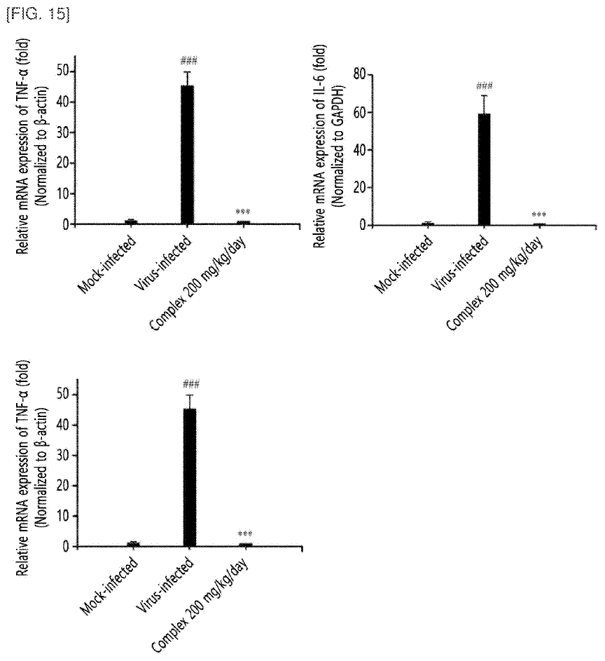

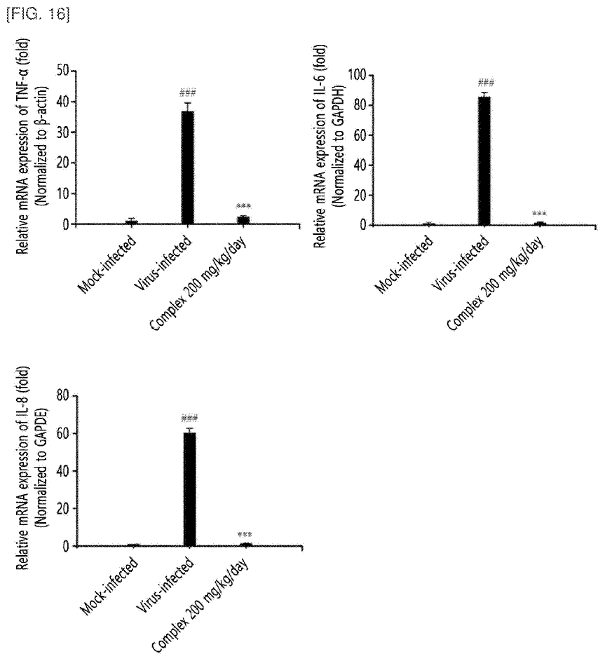

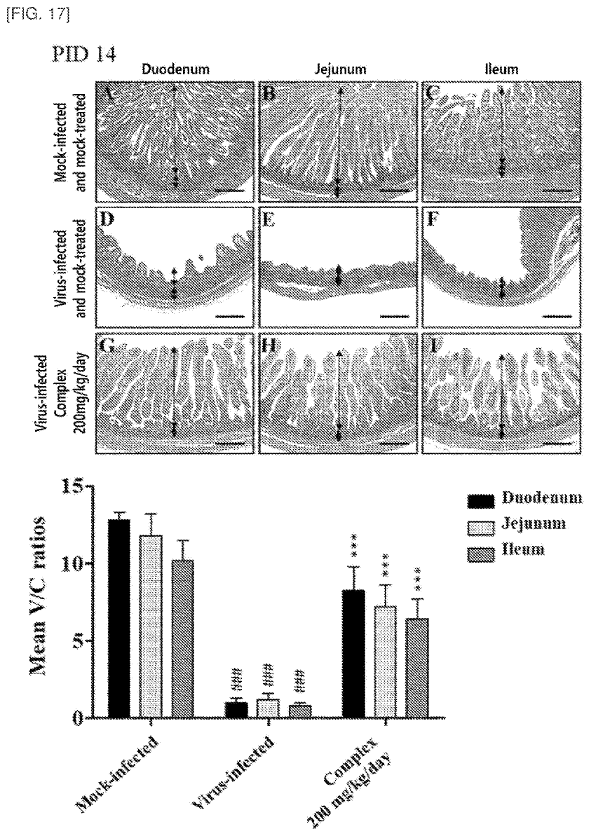

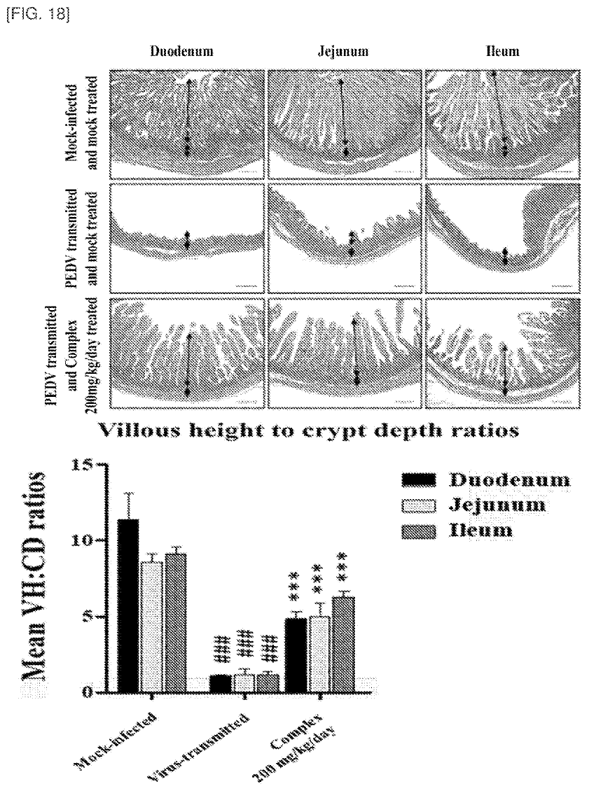

Effects of reducing pro-inflammatory factors in the duodenum of the mock-infected group, the virus-infected group, and the experimental group (raised together and 200 mg/kg/day administered) were examined. As a result, an about 14-fold decrease, an about 19-fold decrease, and an about 5-fold decrease were observed in TNF-α, IL-6, and IL-8, respectively, indicating that the complex has very high anti-inflammatory activity against porcine epidemic diarrhea virus infection (FIG. 13).

3-6-2. Jejunum

Effects of reducing pro-inflammatory factors in the jejunum of the mock-infected group, the virus-infected group, and the experimental group (raised together and 200 mg/kg/day administered) were examined. As a result, an about 21-fold decrease, an about 13-fold decrease, and an about 21-fold decrease were observed in TNF-α, IL-6, and IL-8, respectively, indicating that the complex has very high anti-inflammatory activity against porcine epidemic diarrhea virus infection (FIG. 14).

3-6-3. Ileum

Effects of reducing pro-inflammatory factors in the ileum of the mock-infected group, the virus-infected group, and the experimental group (raised together and 200 mg/kg/day administered) were examined. As a result, an about 45-fold decrease, an about 60-fold decrease, and an about 45-fold decrease were observed in TNF-α, IL-6, and IL-8, respectively, indicating that the complex has very high anti-inflammatory activity against porcine epidemic diarrhea virus infection (FIG. 15).

3-6-4. Mesentery

Effects of reducing pro-inflammatory factors in the mesentery of the mock-infected group, the virus-infected group, and the experimental group (raised together and 200 mg/kg/day administered) were examined. As a result, an about 36-fold decrease, an about 82-fold decrease, and an about 60-fold decrease were observed in TNF-α, IL-6, and IL-8, respectively, indicating that the complex has very high anti-inflammatory activity against porcine epidemic diarrhea virus infection (FIG. 16).

3-7. Gross Clinical Symptoms

The piglets of the negative control group showed no clinical symptoms including diarrhea during the experiment period, and they maintained a normal intestinal morphology and small intestinal barrier. In contrast, the positive control group (virus) continuously showed diarrhea symptoms from day 1 after virus inoculation to the end of the experiment or death. Characteristic gross clinical symptoms were depression, anorexia immediately before/after diarrhea, vomiting, and dehydration and weight loss due to severe watery diarrhea. Characteristic autopsy findings were that the small intestine was filled with fluid, and the contents were visually observed due to gas distension and thinning of the serous membrane. On day 3 after being raised together, the small intestine of the experimental group administered with the complex of 200 mg/kg/day showed a thinner wall of the small intestine and distension due to watery fluid and gas, but maintained the shape of the mucosal folds, similar to the virus-infected group. On day 10 after being raised together, the small intestine of the experimental group administered with the complex of low or high concentrations maintained the normal intestinal morphology and intestinal barrier, and the distinct mucosal folds, similar to the mock-infected group (FIG. 17).

During the experimental period, all the experimental groups except for the negative control group showed lowered intake rates over the period after inoculation. In particular, the positive control group (virus) showed a sharply lowered intake rate from day 3 after being raised together. However, the complex-administered group maintained an intake rate of about 30% or more higher than that of the virus-infected group, indicating that treatment of the complex has the efficacy of improving anorexia and decreased physical strength due to virus infection.

3-8. Histopathological Examination

At the end of the experiment, each individual was euthanized and autopsied, and the small intestine (duodenum, jejunum, ileum) was collected and divided into pieces, fixed in 10% neutral formalin, and subjected to common procedures for preparing pathological tissue samples to prepare Hematoxylin & Eosin stained slides of each tissue, which were used to perform histopathologic examination for villous atrophy of the small intestine. The villous atrophy of the small intestine was compared by obtaining a villi:crypt ratio.

As a result, in the small intestine of the negative control group, the villi projected in the normal finger-like shape, and the crypts were normal in size. In contrast, in the piglets of the positive control group, the virus proliferated in the villus epithelial cells of the small intestine, and the villus epithelial cells were sloughed off. As a result, villous atrophy and fusion occurred, and the proliferation of the crypts to replace the sloughed villi was prominently observed. In addition, hyperemia and hemorrhage were observed in the lamina propria, and mononuclear cell infiltration was observed. As compared with the positive control group, the experimental group treated with the complex of 200 mg/kg/day showed that the overall length of the villi increased, and the villi projected in the normal finger-like shape, and the size of the crypt was also normal (FIG. 18).

As described above, the curcumin-licorice saponin water-soluble complex, in which the water solubility of the curcuminoid-based compound, curcumin was increased using the licorice extract, exhibited excellent inhibitory efficacy against PED coronavirus. Some of the physiological effects of the curcumin alone or the licorice extract (licorice saponin) alone have been reported, but they have no inhibitory effect against PED coronavirus.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are therefore intended to be embraced by the claims.

### Effect of the Invention

A composition including a curcuminoid-based compound and a licorice extract or a fraction thereof according to the present invention exhibits a synergistic effect between these components, and thus the absorption rate in the body is increased, the antiviral effect is remarkably improved, and there are no side effects. Therefore, even though a small amount thereof is used, it may effectively prevent, treat, and ameliorate porcine epidemic diarrhea (PED) virus infection. Accordingly, the composition may be applied to a pharmaceutical composition, a quasi-drug composition, a feed additive, a drinking water additive, a feed, or drinking water for preventing, ameliorating, or treating PED virus infection.

1. A method for preventing or treating porcine epidemic diarrhea (PED) virus infection, said method comprising administering a pharmaceutical composition comprising, as an active ingredient, a complex including a curcuminoid-based compound represented by the following Formula 1 or a pharmaceutically acceptable salt thereof; and a licorice extract or a fraction thereof: 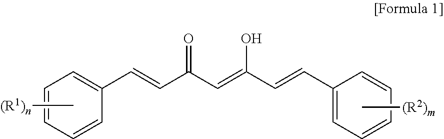
wherein R1 and R2 are each independently hydrogen, a hydroxy group, or a C1-C10 alkoxy group, and
n and m are 1≤n≤5 and 1≤m≤5.

2. The method of claim 1, wherein the curcuminoid-based compound represented by Formula 1 is a compound represented by any one of the following Formulae 2 to 4, or a mixture thereof:

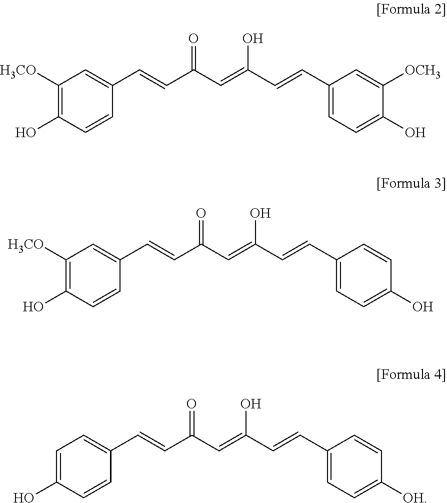 [SMILES: COc1cc(/C=C/C(=O)/C=C(O)/C=C/c2ccc(O)c(OC)c2)ccc1O ; COc1cc(/C=C/C(=O)/C=C(O)/C=C/c2ccc(O)cc2)ccc1O ; O=C(/C=C(O)/C=C/c1ccc(O)cc1)/C=C/c1ccc(O)cc1]

3. The method of claim 1, wherein a weight ratio of the curcuminoid-based compound represented by Formula 1 or the pharmaceutically acceptable salt thereof:the licorice extract or the fraction thereof included in the complex is 1:1 to 1:400.

4. The method of claim 1, wherein the curcuminoid-based compound represented by Formula 1 is isolated from turmeric.

5. The method of claim 1, wherein the pharmaceutical composition is for oral administration.

6. A method for preventing or ameliorating porcine epidemic diarrhea (PED) virus infection, said method comprising administering a food composition comprising, as an active ingredient, a complex including a curcuminoid-based compound represented by the following Formula 1 or a pharmaceutically acceptable salt thereof; and a licorice extract or a fraction thereof: 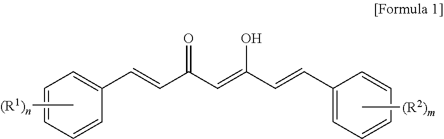
wherein R1 and R2 are each independently hydrogen, a hydroxy group, or a C1-C10 alkoxy group, and
n and m are 1≤n≤5 and 1≤m≤5.

7. The method of claim 6, wherein the curcuminoid-based compound represented by Formula 1 is a compound represented by any one of the following Formulae 2 to 4, or a mixture thereof:

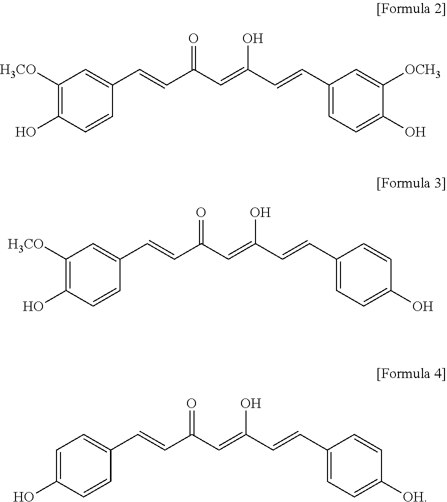 [SMILES: COc1cc(/C=C/C(=O)/C=C(O)/C=C/c2ccc(O)c(OC)c2)ccc1O ; COc1cc(/C=C/C(=O)/C=C(O)/C=C/c2ccc(O)cc2)ccc1O ; O=C(/C=C(O)/C=C/c1ccc(O)cc1)/C=C/c1ccc(O)cc1]

8. The method of claim 6, said composition is a quasi-drug composition.

9. (canceled)

10. The method of claim 6, said composition is a feed additive.

11. (canceled)

12. (canceled)

13. The method of claim 6, said composition is a drinking water additive.

14. (canceled)

15. (canceled)
